(19) United States
(12) Patent Application Publication
(10) Pub. No.: US 2020/0129093 A1
(43) Pub. Date: Apr. 30, 2020

(54) Three-Dimensional Tumor Ruler and Holder

(71) Applicant: Augusta University Research Institute, Inc., Ausgusta, GA (US)

(72) Inventors: Fengchong Kong, North Augusta, SC (US); Jianyue Jin, Indianapolis, IN (US); Christopher Middleton, Augusta, GA (US)

(21) Appl. No.: 16/174,728

(22) Filed: Oct. 30, 2018

(51) Int. Cl. A61B 5/107; G01B 3/04

(52) U.S. Cl. CPC A61B 5/1073; G01B 21/02; A61B 5/1072; G01B 3/04

(57) ABSTRACT

A three-dimensional tumor ruler and holder and methods of use thereof are provided. One embodiment provides a three-dimensional tumor ruler to simultaneously measure the length, width, and height of a tumor when positioned on or near the tumor. The tumor measurements can then be used to calculate tumor volume. Tumor volume can be used to accurately determine for example the dose of a drug or radiation to administer to a subject. In another embodiment, the tumor ruler can be used to position the tumor during administration of a drug or radiation therapy to maximize exposure of the tumor to the therapy.

### CROSS-REFERENCE TO RELATED APPLICATIONS

This application claims benefit of and priority to U.S. Provisional Patent Application No. 62/579,299 filed on Oct. 31, 2017, and is incorporated by reference in its entirety.

### FIELD OF THE INVENTION

The invention is generally directed to devices and methods for measuring and treating tumors.

### BACKGROUND OF THE INVENTION

To effectively study and treat tumors, it is important to know the volume of the tumor. Various formulas utilizing height (h), length (l), and width (w) calculate the volume (V) of the tumor. For example, V=(hlw)/2 and V=πhlw/6 are common formulas that have been used to calculate tumor volume.

However, capturing the needed dimensions can be difficult. For example, tumors on research mice, small in size, can require numerous attempts to capture the dimensions. Further, capturing measurements via caliper rulers can lead to less accurate numbers given that the tumors are soft tissue, allowing the calipers to squeeze the tissue and capture dimensions smaller than the actual size of the tumor. In many cases, to save time and effort, researchers will assume that the width and the height of the tumor are the same; such an assumption leads to significant inaccuracies in the calculation of the size of the tumor.

Tumor measurements are used for determining the appropriate drug or radiation dosage to effectively treat the tumors. To ensure appropriate administration of the drug or radiation, the location and size of the tumor must be determined to expose the whole tumor, and only the tumor, to radiation. It is important to minimize exposure of healthy tissue to chemotherapy or radiation.

Therefore, there is a need for a device that allows easy and accurate measurement of the dimensions of a tumor to calculate tumor volume. In addition, there is also a need for a device that can be used to assist in radiation treatment of the tumor.

### SUMMARY OF INVENTION

A three-dimensional tumor ruler and holder and methods of use thereof are provided. In one aspect, the three-dimensional tumor ruler is used to simultaneously measure the length, width, and height of a tumor when positioned on or near the tumor. The tumor measurements can then be used to calculate tumor volume.

In another aspect, the three-dimensional tumor ruler is used to position the tumor during administration of a drug or radiation therapy to maximize exposure of the tumor to the therapy. The three-dimensional ruler/holder fully exposes the tumor to drug or radiation therapy and can block portions of the drug or radiation therapy from healthy tissue or organs at risk (OAR) and non-targeted portions of the subject's body.

### BRIEF DESCRIPTION OF THE DRAWINGS

FIG. 1 is a top plan view of a three-dimensional tumor ruler and holder according to an aspect of the present invention.

FIG. 2 is a bottom plan view of the three-dimensional tumor ruler and holder of FIG. 1

FIG. 3 is a front plan view of the three-dimensional tumor ruler and holder of FIG. 1

FIG. 4 is a back plan view of the three-dimensional tumor ruler and holder of FIG. 1

FIG. 5 is a left plan view of the three-dimensional tumor ruler and holder of FIG. 1

FIG. 6 is a right plan view of the three-dimensional tumor ruler and holder of FIG. 1.

### DETAILED DESCRIPTION OF THE EMBODIMENTS

### I. Tumor Measurement Devices

In the following detailed description of the preferred embodiments, reference is made to the accompanying drawings, which form a part hereof, and within which are shown by way of illustration specific embodiments by which the invention may be practiced. It is understood that other embodiments may be utilized and structural changes may be made without departing from the scope of the invention.

One embodiment is directed to a three-dimensional tumor ruler and holder 100. The three-dimensional (3D) tumor ruler and holder 100 provides a means for accurately measuring a given tumor in one application. The 3D tumor ruler and holder 100 can be used to position the tumor to maximize administration of a drug or radiation to the tumor.

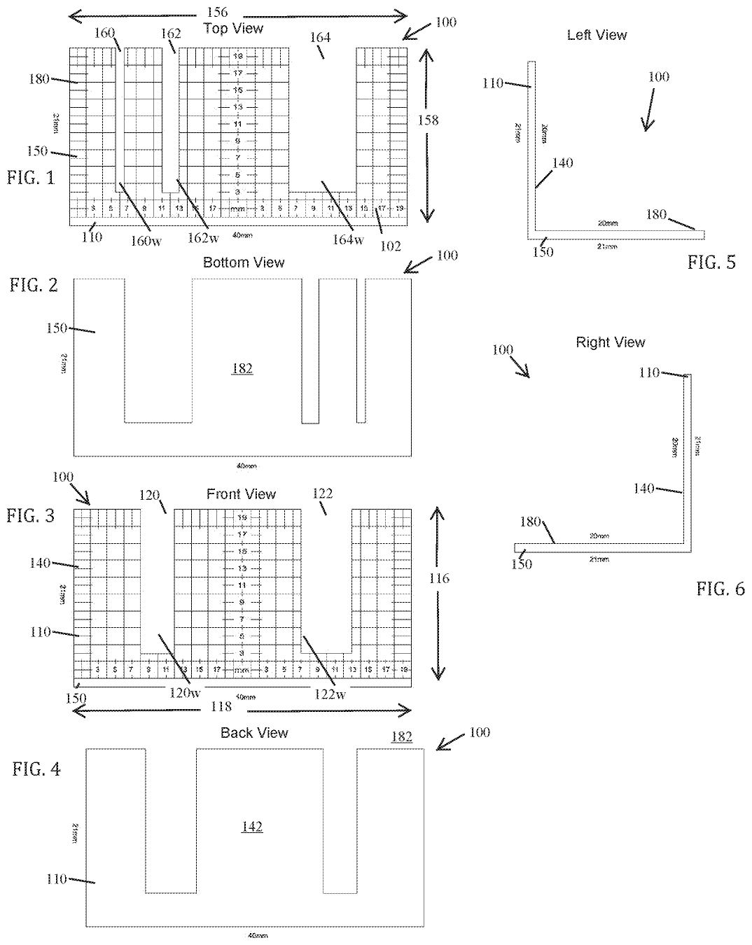

FIGS. 1-6 illustrate the 3D tumor ruler and holder 100 according to an aspect of the present invention. The 3D tumor ruler and holder 100 includes a body 102 formed from a vertical member 110 and a horizontal member 150. As shown, the vertical member 110 and the horizontal member 150 are joined together, forming substantially a right angle (see FIGS. 5-6), allowing for accurate measurements of the height, width, and length of the targeted tumor. In an aspect, the vertical member 110 and the horizontal member 150 are formed from a single piece. In an aspect, the holder 100 can be made of a variety of different materials. For example, the material of the body 102 can include, but is not limited to known wood, plastics, various metals, resins and radiation-shielding materials. In an aspect, the body 102 can be made of a transparent material. The ruler 100 may be made from any suitable material such as wood, metal, aluminum, carbon fiber, titanium, plastic, resins and any combination thereof. The ruler 100 may be made from a material that maintains rigidity and retains accuracy.

The vertical member 110 and the horizontal member 150 both have respective measurement surfaces 140, 180, discussed in detail below, as well as back surfaces 142, 182. The measurement surfaces 140, 180 of the vertical and horizontal members 110, 150 are configured to be adjacent to one another, assisting in providing the measurement of the targeted tumor. Ruler 100 contains measurement elements. As used throughout this description, a measurement element is any type of element, such as a printed marking, protrusion, recess, indent, etc., that identifies one location in relationship to another location. For example, a measurement element may be a protrusion of a different color on the measurement surface 140 marking how far one point is from the measuring surface 180 of the horizontal member 150 of the ruler 100. In some embodiments, the measurement elements are embossed, and/or debossed. The measurement elements may identify any units of measure helpful to the user. For example, the measurement elements can be in millimeter increments, with larger increments, such as at each centimeter, labeled with numbers.

As shown in FIG. 3, the vertical member 110 has a height 116 and a width 118. Similarly, the horizontal member 150 includes a width 156 and a length 158. The height 116 and the width 118 of the vertical member 110 and the width 156 and length 158 of the horizontal member can vary depending on the application of the 3D tumor ruler and holder 100. For example, the 3D tumor ruler and holder 100 of FIGS. 1-6 is configured to measure exterior tumors found on research mice, so the holder 100 is relatively small in dimension (e.g., width 118, 156 is approximately 40 mm, height 116 is approximately 21 mm, and the length 158 is approximately 21 mm). In other applications, the width 118, 156, height 116, and length 158 can vary based upon the target of measurement. As shown in FIGS. 5-6, the length 158 and the height 116 of the measurement surfaces 140, 180 of the vertical and horizontal members 110, 150 are less than that of the overall members 110, 150 due to the fact of the intersection of the members 110, 150.

As shown in FIGS. 1-2, the horizontal member 150 includes tumor-receiving slots 160, 162, and 164. The horizontal member 150 includes three tumor-receiving slots 160, 162, 164, with each slot having its own width 160w, 162w, 164w. The widths 160w, 162w, 164w vary from one another in order to provide slots that can receive various sized tumors but maintain accuracy during measurement. While FIGS. 1-2 show only three tumor-receiving slots 160, 162, 164, other embodiments of the present invention can vary in the number of tumor-receiving slots. As discussed above, the 3D tumor ruler and holder 100 is configured to receive tumors located on the exterior of the subject. However, in other embodiments, the 3D tumor ruler and holder 100 can be used on tumors located within the body of the subject as long as those tumors are accessible (e.g., during surgery).

As shown in FIGS. 3-4, the vertical member 110 includes two viewing slots 120, 122. In other aspects, the number of viewing slots can vary. The viewing slots 120, 122 provide access for a user to see the tumors from the opposite side, as well as provide a window for radiation treatment, while shielding other portions of the body of the subject with the tumor. Similar to the tumor-receiving slots 160, 162, 164, the viewing slots 120, 122 can vary in width 120w, 122w in order to provide a full window for radiation treatment of tumors of various sides. In other words, the smaller viewing slot 120 is used for viewing and treating smaller tumors, and the larger viewing window 122 is used for larger-sized tumors. In an aspect, the viewing slots 120, 122 are substantially aligned with the tumor-receiving slots 160, 162, 164. As shown in FIGS. 1-4, the smaller tumor-receiving slots 160, 162 can be aligned with the smaller viewing slot 120, and the larger tumor-receiving slot 164 can be aligned with the larger viewing slot 122.

As discussed above, the vertical member 110 and the horizontal member 150 have corresponding measuring surfaces 140, 180. The measuring surfaces 140, 180 can have markings corresponding to dimensions in two planar directions—height and width on the vertical measuring surface 140, and length and width on the horizontal measuring surface 180. As shown in the figures, the unit of measure utilized is in millimeters, with score lines utilized to indicate the dimension at a particular point. However, in other embodiments, various other units of measure for length (cm, in, etc.) can be used. By including all three dimensions in one viewable field, the 3D tumor ruler and holder 100 allows the user to quickly measure the size of the tumor, capturing the height, width, and length. From these dimensions, accurate calculations of the volume of the tumor are performed.

As discussed above, the 3D tumor ruler and holder 100 is utilized to position the tumor to maximize administration of drug or radiation therapy. In many instances, cone beam computed tomography (CBCT) is used to help identify the tumor. However, tumor edges inside the body are hard to identify. To overcome this shortcoming, the prior art has used the application of contrast agents to the tumor. In an aspect, the holder 100, via the slots 160, 162, 164, helps show the edges of the tumor, which allows the entire tumor to be identified without the need for contrasts, saving time and money. In addition, the slots 160, 162, 164 can pull the tumor up from the skin of the subject, which allows the application of drug or radiation therapy to the tumor only. In addition, the therapy can be applied through the viewing slots 120, 122, which further shield the healthy tissue of the subject.

### II. Methods of Use

One embodiment provides a method of treating a tumor by determining the dimensions of a tumor using the 3D tumor ruler and holder 100 on a tumor, calculating the volume of the tumor using the measuring surfaces of the ruler and holder 100, and administering a treatment to the tumor based on the calculated volume of the tumor to kill tumor cells in the tumor.

The disclosed ruler and holder 100 can be used to calculate the volume of a variety of tumors. The term “tumor” refers to a neoplasm. A neoplasm is an abnormal new growth of cells. The cells in a neoplasm usually grow more rapidly than normal cells and will continue to grow if not treated. As they grow, neoplasms can impinge upon and damage adjacent structures. The term neoplasm can refer to benign (usually curable) or malignant (cancerous) growths.

In one embodiment, an effective amount of a chemotherapeutic agent is administered to the tumor in a dose based on the calculated volume of the tumor. Representative chemotherapeutic agents include, but are not limited to bifunctional alkylators, monofunctional alkylators, anthracyclines, cytoskeletal disruptors, epothilones, histone deacetylase inhibitors, inhibitors of topoisomerase I, inhibitors of topoisomerase II, kinase inhibitors, nucleotide analogs and precursor analogs, peptide antibiotics, platinum-based agents, and retinoids. Specific examples of chemotherapeutic agents include, but are not limited to actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and vinorelbine.

In another embodiment, the therapy is radiation therapy. Radiation therapy uses high-energy radiation to shrink tumors and kill cancer cells (Lawrence T S, Ten Haken R K, Giaccia A. Principles of Radiation Oncology. In: DeVita V T Jr., Lawrence T S, Rosenberg S A, editors. Cancer: Principles and Practice of Oncology. 8th ed. Philadelphia: Lippincott Williams and Wilkins, 2008). X-rays, gamma rays, and charged particles are types of radiation used for tumor treatment. The radiation may be delivered by a machine outside the body (external-beam radiation therapy), or it may come from radioactive material placed in the body near cancer cells (internal radiation therapy, also called brachytherapy).

In one embodiment, the tumor is treated with an effective amount of a radiosensitizer followed by treatment with radiation. Radiosensitizers are drugs that make tumor cells more sensitive to the effects of radiation therapy. Exemplary radiosenstizers include, but are not limited to carbogen, nicotinamide, metronidazole, misonidzole, etanidazole, nimorazole, mitomycin-C, tirapazamine, procaine, lidocaine, chlorpromazine, bromodeoxyuridine, iododeoxyuridine, hydroxyurea, gemcitabine, and fludarabine. In addition, some anticancer drugs, such as 5-fluorouracil and cisplatin, make cancer cells more sensitive to radiation therapy.

In another embodiment, the 3D tumor ruler and holder 100 is used to position the tumor for surgical extraction. The 3D tumor ruler and holder 100 can be placed around the tumor, via one of the tumor receiving slots 160, 162, 164. From here, the user can then remove the tumor, with the 3D tumor ruler and holder 100 protecting other portions of the subject's body.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, those skilled in the art will appreciate that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A three-dimensional tumor ruler and holder comprising:
a. a vertical member comprising:
i. a vertical measurement surface for measuring a height and width of a targeted tumor; and
ii. at least one viewing slot;
b. a horizontal member connected to the vertical member, forming a right angle, the horizontal member comprising:
i. a horizontal measurement surface for measuring the width and length of the targeted tumor; and
ii. at least one tumor-receiving slot, wherein the at least one tumor-receiving slot is configured to slide around the targeted tumor.

2. The three-dimensional tumor ruler and holder of claim 1, wherein the at least one tumor-receiving slot comprises a plurality of tumor-receiving slots, wherein each tumor-receiving slot has a unique width, wherein the unique widths allow for the plurality of tumor-receiving slots to receive a variety of different sized targeted tumors.

3. A method for determining the volume of a targeted tumor comprising:
a. providing three-dimensional tumor ruler comprising:
i. a vertical member comprising:
I. a vertical measurement surface for measuring a height and width of the targeted tumor; and
II. at least one viewing slot;
ii. a horizontal member comprising:
I. a horizontal measurement surface for measuring the width and length of the targeted tumor; and
II. at least one tumor-receiving slot, wherein the at least one tumor-receiving slot is configured to slide around the targeted tumor;
b. sliding the targeted tumor into the at least one tumor-receiving slot; and
c. observing the height, width, and length of the targeted tumor by looking at the horizontal and vertical measurement surfaces of the three-dimensional tumor ruler.

4. A method for treating a targeted tumor on a subject comprising:
a. providing three-dimensional tumor holder comprising:
i. a vertical member comprising at least one viewing slot;
ii. a horizontal member comprising at least one tumor-receiving slot, wherein the at least one tumor-receiving slot is configured to slide around the targeted tumor;
b. sliding the targeted tumor into the at least one tumor-receiving slot; and
c. applying radiation through the at least one viewing slot on the targeted tumor.
